(19) United States
(12) Patent Application Publication
(10) Pub. No.: US 2008/0167680 A1
(43) Pub. Date: Jul. 10, 2008

(54) Fingertip Surgical Instrument

(76) Inventors: James W. Voegele, Cincinnati, OH (US); Robert P. Gill, Mason, OH (US); Wayne L. Poll, New Albany, OH (US)

Correspondence Address: FROST BROWN TODD, LLC, 2200 PNC CENTER, 201 E. FIFTH STREET, CINCINNATI, OH, 45202, US

(21) Appl. No.: 11/621,702

(22) Filed: Jan. 10, 2007

(51) Int. Cl. A61B 17/29

(52) U.S. Cl. 606/206

(57) ABSTRACT

Disclosed is a minimally invasive surgical instrument that may be used in hand-assisted laparoscopic surgeries. The device is a multifunctional surgical instrument that may be mounted directly on a surgeon's fingertip and inserted through an incision to allow the surgeon to manipulate tissue during a surgical procedure. Versions show a self-righting suture needle holder and a version with a dual pivoting jaw (e.g., grasper, cutter) that has an elongate neck to enhance the reach of the instrument. In addition, an elongate neck of an actuated fingertip instrument enhances the range reachable through a single incision.

### CROSS REFERENCE TO RELATED APPLICATIONS

The present application is related to and claims the benefit of two commonly-owned U.S. patent applications (a) Ser. No. 11/533,506, entitled “Dispensing Fingertip Surgical Instrument” to Voegele et al. filed on Sep. 20, 2006; and (b) U.S. patent application Ser. No. 10/777,324, “Fingertip Surgical Instruments” to Voegele et al., filed Feb. 12, 2004, published as U.S. patent application Publ. No. 2004/0193211 A1 on Sep. 30, 2004, the disclosures of which are hereby incorporated by reference in their entirety.

The present application is further related to three U.S. patent applications: (1) U.S. patent application Ser. No. 10/777,740, “Fingertip Ultrasound Medical Instrument” to Voegele et al., filed Feb. 12, 2004, published Nov. 11, 2004 as U.S. patent application Publ. No. 2004/0225217 A1; (2) U.S. patent application Ser. No. 10/777,708, “Multifunctional Surgical Instrument” to Voegele et al., filed Feb. 12, 2004, published Oct. 7, 2004 as U.S. patent application Publ. No. 2004/0199204 A1; and (3) U.S. patent application Ser. No. 11/398,985 “A Multi-Port Insert For Use With A Laparoscopic Access Device” to Voegele et al., filed Apr. 5, 2006, which in turn claims the benefit of U.S. patent application Ser. No. 60/669,514 filed Apr. 8, 2005, the disclosures of all of which are hereby incorporated by reference in their entirety.

### FIELD OF THE INVENTION

The present invention relates, in general, to minimally invasive surgical instruments that may be used in hand-assisted laparoscopic surgeries where the instruments are mounted directly on a surgeon's fingertip and are then inserted through an incision, perhaps pneumatically sealed with a laparoscopic disk, to allow the surgeon to manipulate internal tissue during a surgical procedure.

### BACKGROUND OF THE INVENTION

Abdominal surgery typically involves an incision in the abdominal wall large enough to accommodate a surgeon's hands, multiple instruments, and illumination of the body cavity. While large incisions simplify access to the body cavity during a surgery, it also increases trauma, requires extended recovery time, and can result in unsightly scars. In response to these drawbacks, minimally invasive surgical methods have been developed.

In minimally invasive abdominal surgery, or laparoscopic surgery, several smaller incisions are made into the abdominal wall. One of the openings is used to inflate the abdominal cavity with gas, which lifts the abdominal wall away from underlying organs and provides space to perform the desired surgery. This process is referred to as insufflation of the body cavity. Additional openings can be used to accommodate cannulas or trocars for illuminating and viewing the cavity, as well as instruments involved in actually performing the surgery, e.g., instruments to manipulate, cut, or resect organs and tissue.

While minimally invasive surgical methods overcome certain drawbacks of traditional open surgical methods, there are still various disadvantages. In particular, there is limited tactile feedback from the manipulated tissue to the surgeon hands. In non-endoscopic surgery, a surgeon can easily verify the identification of structures or vessels within a conventional open surgery incision. In particular, the surgeon normally uses the sense of feel to verify the nature of visually identified operational fields. Further, in endoscopic surgery, tissue that is to be removed from the body cavity must be removed in pieces that are small enough to fit through one of the incisions.

Recently, new surgical methods have been developed that combine the advantages of the traditional and minimally invasive methods. It is sometimes referred to as hand assisted laparoscopic surgery (“HALS”). In these new methods, small incisions are still used to inflate, illuminate, and view the body cavity, but in addition, an intermediate incision is made into the abdominal wall to accommodate the surgeon's hand. The intermediate incision must be properly retracted to provide a suitable-sized opening, and the perimeter of the opening is typically protected with a surgical drape to prevent bacterial infection. A sealing mechanism is also required to prevent the loss of insufflation gases while the surgeon's hand is either inserted into or removed from the body cavity though the retracted incision.

While the hand provides a great deal of flexibility and retains the surgeon's sense of feel, fingers in themselves have limits as to their usefulness. Fingers lack the delicacy to pick up fine tissue. Fingers require making larger divisions when dissecting tissue. Fingers are subject to injury when holding tissue while energy modalities, such as ultrasound or RF, are used to treat the surgical site. Traditional instruments intended for conventional surgery, i.e. forceps and graspers, are too large for the limited body cavity environment. Traditional instruments also present the problem of being brought into and out of the laparoscopic site causing time-delaying deflation and re-insufflations of the body cavity. Laparoscopic equivalent instruments are delivered through a body wall port and have limited access to tissue. U.S. Pat. Nos. 6,149,642; and 5,925,064 disclose various aspects of laparoscopic surgery and fingertip devices for surgeon use.

With the advance represented by HALS procedures, there is a need for improved fingertip surgical instrumentation that can take advantage of the increased freedom created by having a hand inside the body cavity.

As an example, while suturing is often necessary in HALS procedures, generally known suturing instruments may prove difficult when repetitively gripping a curved suture needle and forming a stitch.

As yet a further example, while instruments for HALS necessarily need to be of limited dimensions in order to be effective within the close confines of the insufflated abdomen, some additional range of motion is often desirable.

Consequently, a significant need exists for an improved surgical instrument useful in HALS procedures.

### BRIEF SUMMARY OF THE INVENTION

The invention overcomes the above-noted and other deficiencies of the prior art by providing a fingertip surgical instrument that has an end effector that may be efficiently actuated with one hand within the close confines of an insufflated body cavity in order to manipulate internal tissue.

In one aspect of the invention, a surgical instrument includes a fingertip mounting structure that is attachable to a fingertip of a surgeon so that his hand may be inserted through an incision to perform a hands assisted laparoscopy surgery (HALS) procedure, specifically suturing with an end effector having a pair of jaws with opposing generally convex and concave registering surfaces that tend to properly orient (i.e., self righting) a curved suture needle.

In another aspect of the invention, a surgical instrument includes a fingertip mounting structure that is attachable to a fingertip of a surgeon so that his hand may be inserted through an incision to perform a hands assisted laparoscopy surgery (HALS) procedure. A pair of pivotal jaws are coupled by an elongate neck to the finger mounting portion so that an increased range of motion is achieved. A finger actuated member affects opening and closing the pair of pivotal jaws.

These and other objects and advantages of the present invention shall be made apparent from the accompanying drawings and the description thereof.

### BRIEF DESCRIPTION OF THE FIGURES

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1 is a cut-away perspective view of an exemplary use of a fingertip surgical instrument attached to a gloved finger of a surgeon's hand inserted through a laparoscopic disk into an insufflated abdomen of a patient undergoing Hand Assisted Laparoscopy Surgery (HALS).

FIG. 2 is a left front perspective view of a self-righting needle holder, as a first illustrative version of the fingertip surgical instrument of FIG. 1.

FIG. 3 is an exploded view of the self-righting needle holder of FIG. 2 from a position above, left and in front.

FIG. 4 is an exploded view of the self-righting needle holder of FIG. 2 from a position below, left and in front.

FIG. 5 is left side view taken in vertical longitudinal cross section through the self-righting needle holder of FIG. 2 mounted on an index finger and having a thumb slide positioned distally to close and lock an upper jaw.

FIG. 6 is left side view taken in vertical longitudinal cross section through the self-righting needle holder of FIG. 2 mounted on an index finger and having a thumb slide positioned proximally to unlock and open the upper jaw.

FIG. 7 is a left side view in vertical longitudinal cross section through an elongate grasper in an unactuated, closed position, as a second illustrative version of the fingertip surgical instrument of FIG. 1.

FIG. 8 is a left side view in vertical cross section of the elongate grasper of FIG. 7 in an actuated, open position.

FIG. 9 is a left side view in elevation of yet an additional fingertip surgical instrument cut away to depict an extending inkpad for marking internal tissue actuated by pressure on an end effector nozzle.

FIG. 10 is a left side view in elevation of yet another alternative fingertip instrument partially cut away to expose a roller ball that applies ink to internal tissue.

FIG. 11 is a left side view in elevation of a further alternative fingertip surgical instrument partially cut away to expose a marking element extending from a finger mounted end effector tip that is unsheathed for use by distally advancing a thumb actuator.

FIG. 12 is a left side view in elevation of an additional alternative fingertip surgical instrument partially cut away to expose a marking element distally extending from a thumb actuator that is extended out of a fingertip mounted end effector nozzle for use.

FIG. 13 is a left side view in elevation of yet another alternative fingertip surgical instrument partially cut away to expose a marking solid element urged by a spring biased plunger into contact with a convex porous tip for marking internal tissue.

FIG. 14 is a left side view in elevation of another alternative fingertip surgical instrument partially cut away to expose a thumb actuator that longitudinally compresses an ampoule for piercing and dispensing flowable material out of a nozzle tip.

FIG. 15 is a left side view in elevation of yet a further alternative fingertip surgical instrument partially cut away to expose an upwardly pivoting thumb actuator that crushes an ampoule for dispensing flowable materials.

FIG. 16 is a front view taken in cross section along lines 16-16 of FIG. 15 of the further alternative fingertip surgical instrument of FIG. 15.

FIG. 17 is a left side view in elevation of an additional alternative fingertip surgical instrument partially cut away to expose an upwardly pivoting thumb actuator that raises a linearly-moved actuator member to crush an ampoule and to dispense flowable material.

FIG. 18 is a left side view in elevation of another alternative fingertip surgical instrument partially cut away to expose a thumb actuator in an initial position prior to dispensing.

FIG. 19 is a left side view in elevation of the fingertip surgical instrument of FIG. 18 partially cut away to expose the thumb actuator having been depressed to break an ampoule.

FIG. 20 is a left side view in elevation of the fingertip surgical instrument of FIG. 18 partially cut away to expose the thumb actuator after distal advancement followed by further upward depression to dispense in a controlled fashion the flowable liquid released from the ampoule.

FIG. 21 is a left side view in elevation of an alternative fingertip surgical instrument for dispensing necked ampoules.

FIG. 22 is a left side view in elevation of the alternative fingertip surgical instrument of FIG. 21 mounted on an index finger and partially cut away to expose a two-compound necked ampoule prior to dispensing.

FIG. 23 is a left side view in elevation of the alternative fingertip surgical instrument of FIG. 22 partially cut away to expose the two-compound necked ampoule after dispensing.

FIG. 24 is a left side view in longitudinal vertical cross section through the two-compound necked ampoule of FIG. 22.

FIG. 25 is a left side view in longitudinal vertical cross section through an alternative single compound necked ampoule for the alternative fingertip surgical instrument of FIG. 22.

FIG. 26 is a left side view of yet another alternative fingertip surgical instrument for dispensing a larger quantity of two flowable materials mixed during dispensing with an upper finger holder cut away.

FIG. 27 is a top view of a distal portion of the fingertip surgical instrument in horizontal cross section along lines 27-27 through a longitudinal centerline.

FIG. 28 is an aft view taken in cross section along lines 28-28 of the fingertip surgical instrument of FIG. 27 through proximal cylindrical vessels, each containing one of the two flowable materials.

FIG. 29 is an aft view taken in cross section along lines 29-29 of the fingertip surgical instrument of FIG. 27 viewing manifolds and a central nozzle tube for mixing and dispensing the two flowable materials.

FIG. 30 is a left side view in elevation of the fingertip surgical instrument of FIG. 26 after actuation of the upper fingertip holder and opposing lower thumb actuator.

FIG. 31 is a top view in cross section taken along lines 31-31 of the fingertip surgical instrument of FIG. 30 after actuation and dispensing.

### DETAILED DESCRIPTION OF THE INVENTION

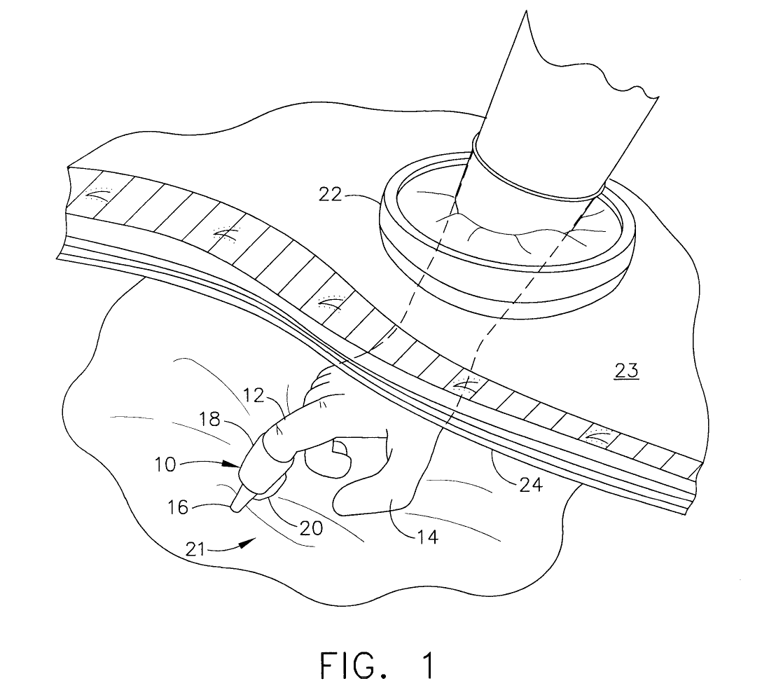

Referring now to FIG. 1, the environment for performing an endoscopic surgical procedure within an abdomen is illustrated, herein referred to as Hand Assisted Laparoscopic Surgery (HALS). A surgeon places a fingertip instrument 10 consistent with aspects of the present invention on his index finger 12 (although any finger can be used) of a gloved hand 14. In particular, the fingertip instrument 10 includes an end effector (working element) 16 distally mounted on a finger tip attachment portion 18 with an actuator 20 movably attached thereto that is moved to actuate the end effector 16 to manipulate tissue 21 (e.g., dispense, mark, activate, pivot, scissor, grasp, etc.). A means for providing hand access, such as a lap disc 22, for example, model LD111 available from Ethicon Endo-Surgery, Cincinnati, Ohio, is placed into an abdominal wall 23 to serve as a pressure seal. The surgeon inserts his arm and gloved hand 14 through the lap disc 22 and into an insufflated abdominal cavity 24.

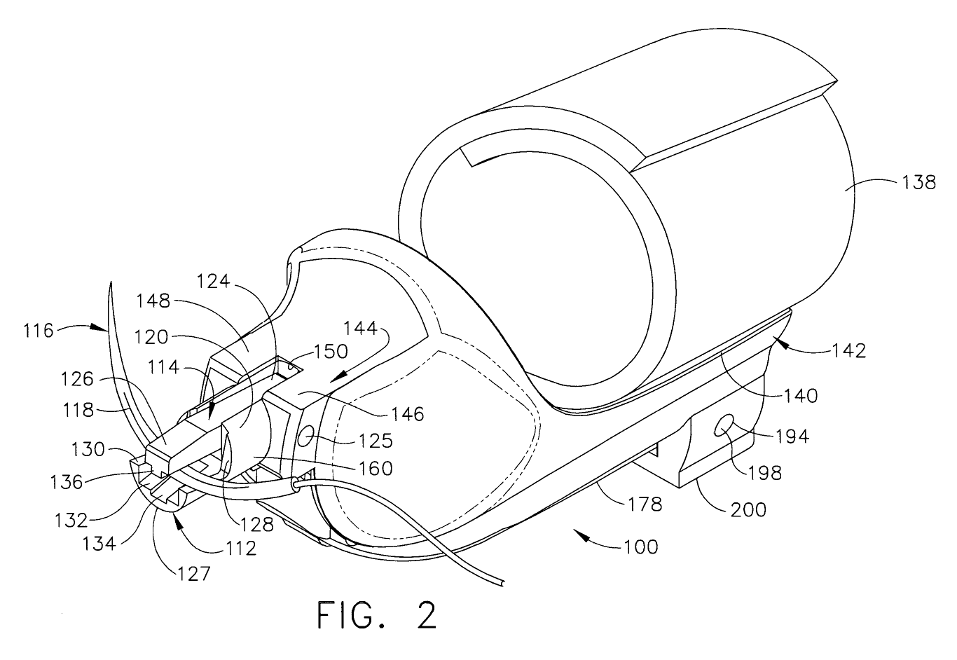

Needle Holder. In the afore-mentioned U.S. patent application Publ. No. 2004/0193211 A1, a needle holder was disclosed as one illustrative working element. In FIGS. 2-6 a version of the fingertip instrument 10 is depicted as a needle holder 100 advantageously including a lower jaw 112 and pivotally attached upper jaw 114 that advantageously form a self-righting grip to a curved suture needle 116 (FIG. 2) having a flattened surface 118 on its concave side. The lower jaw 112 has a proximal cylindrical portion 120 with a vertically and proximally open slot 122 formed therein to receive a proximal rocker portion 124 of the upper jaw 114 that is pinned by a small horizontal pin 125 therein for pivotal opening and closing of a distal end 126 toward the lower jaw 112.

In particular, a distal end 127 of the lower jaw 112 has a cylindrical solid shape with an upper distal removed portion 128 to form a lower contact tray surface 130 into which a rectilinear ramped recess 132 is formed into which in turn a deeper but narrower rectilinear ramped recess 134 is formed. The distal end 126 of the upper jaw 114 has a width easily accommodated by the rectilinear ramped recess 132. The distal end 126 of the upper jaw 114 has a downwardly projecting longitudinal squared off ridge 136 that has a width easily accommodated within the deeper but narrower rectilinear ramped recess 134. Thus, the distal end 126 of the upper jaw 114 has a T-shape in transverse cross section that interacts with the recess 132 in the lower jaw 112 to tend to roll a loosely gripped suture needle 116 toward an upright position with an increased grip. This capability facilitates efficient suturing within the close confines of a HALS procedure. It should be appreciated that the recesses 132, 134 and the ridge 136 may be curved surfaces rather than squared off. Any shape that allows a downwardly projecting portion to be within a recessed area would serve to orient a suture needle 16.

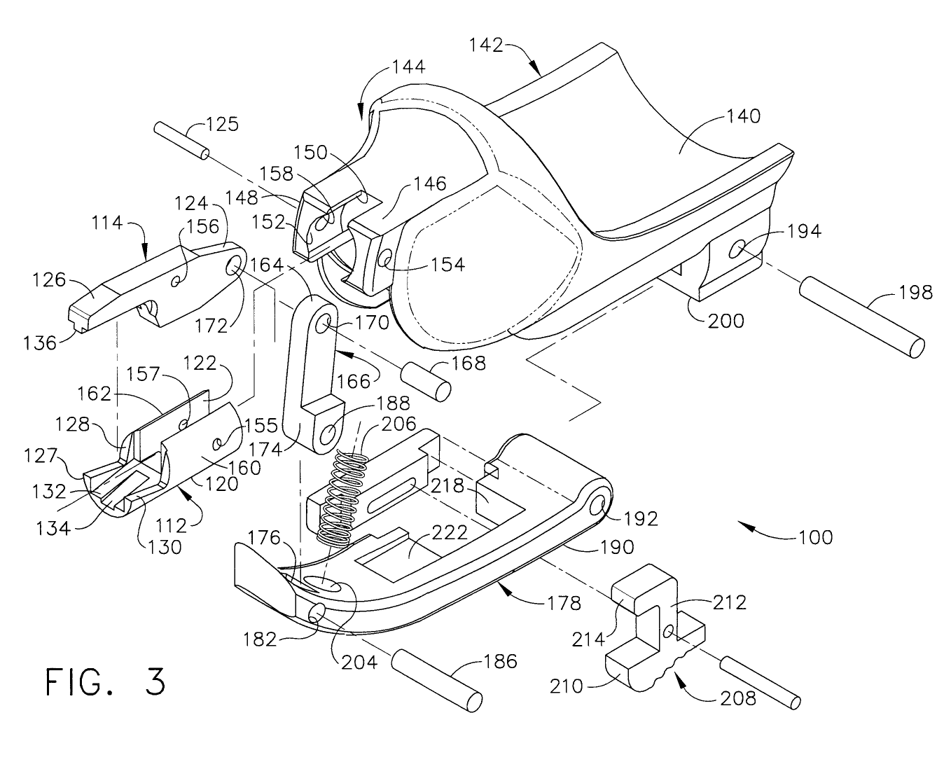

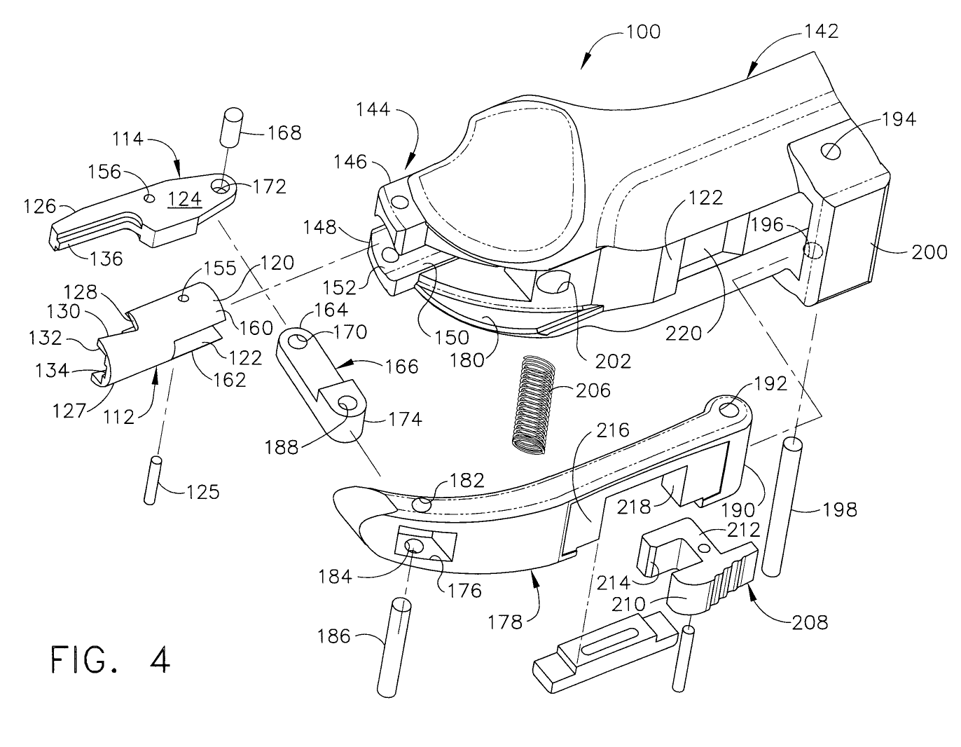

A wing or band 138 of adhesive tape (e.g., cloth surgical tape) or hook loop material (e.g., VELCRO) provides a finger tip attachment portion to secure a curved upper, proximal surface 140 of a sled-shaped finger holder 142 to an undersurface and fingertip of the last digit of the index finger 12. It should be appreciated that the wing or band 138 may be formed from various types of adjustable attachment means that would be acceptable for surgical use and that may securely grip the finger 12. With particular reference to FIGS. 3-4, In addition to being attachable to the finger 12 for movement, the finger holder 142 supports components that are moveably attached for closing the jaws 112, 114 and for locking the jaws 112, 114 closed. A distal snout 144 includes left and right distal mounting arms 146, 148 separated by a vertical slot 150 that widens into a longitudinal, distally open cylindrical recess 152 that receives the proximal cylindrical portion 120 of the lower jaw 112, secured by pin 125 received respectively through five holes 154-158 formed in the left distal mounting arm 146, a left arm 160 of the proximal cylindrical portion 120, the proximal rocker portion 124 of the upper jaw 114, a right arm 162 of the proximal cylindrical portion 120, and the right distal mounting arm 148.

The proximal rocker portion 124 of the upper jaw 114 rotates up and down along with an upper end 164 of a closure link 166 pinned for pivoting movement on the right with an upper pin 168 through right and left holes 170, 172 respectively. A lower end 174 of the closure link 166 pivots within a front vertical slot 176 formed in a curved underslung actuating member 178 received within an elongate recess 180 formed in the undersurface of the sled shaped finger holder 142. Left and right front horizontal through holes 182, 184 formed in the actuating member 178 communicate with the front vertical slot 176 to receive a front horizontal pin 186 that also passes through a bottom hole 188 in the lower end 174 of the closure link 166. An aft end 190 of the underslung actuating member 178 has a proximal horizontal through hole 192 that is aligned with left and right proximal holes 194, 196 (FIG. 4) to pivotally receive a proximal horizontal pin 198, the holes 194, 196 formed in a U-shaped bracket 200 attached to a proximal underside of the sled shaped finger holder 142.

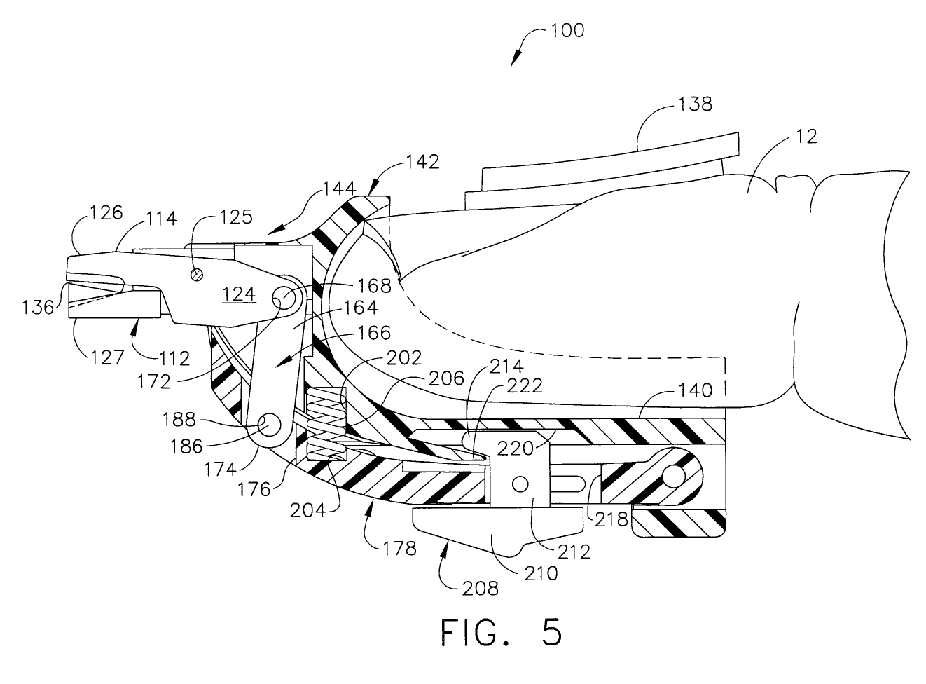

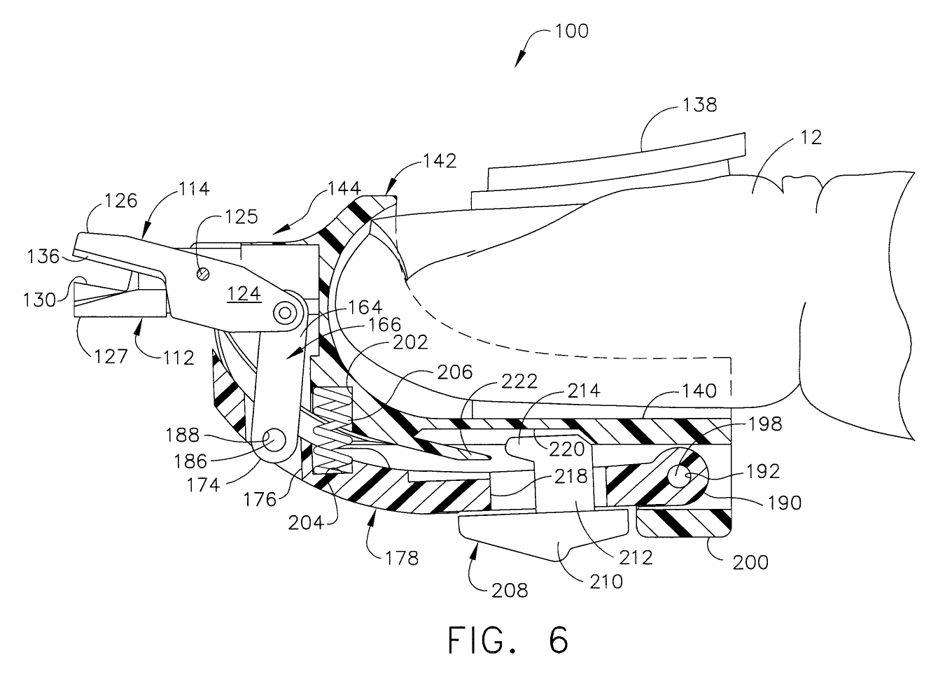

A downwardly open spring recess 202 (FIG. 4) formed in the elongate recess 180 in the sled-shaped finger holder 142 is aligned with an upwardly open spring recess 204 (FIG. 3) formed in the curved underslung actuating member 178 to receive a compression spring 206 that urges the actuating member 178 away from the finger holder 142 when allowed, drawing the closure link 166 to open the upper jaw 114 (FIG. 6). Closure and locking of the upper jaw 114 is effected by distal movement of a thumb slide 208. In particular, the thumb slide 208 has a lower thumb slide button portion 210 attached to an upward arm 212 having a locking tip 214 extending distally from a top end. A shallow rectangular recess 216 (FIG. 4) is sized to contact an upper surface of the low thumb slide button portion 210 allowing some longitudinal sliding. A rightwardly open aperture 218 longitudinally centered in the shallow rectangular recess 216 is sized to allow the upward arm 212 to move a like amount fore and aft with the locking tip 214 received within a downwardly open locking recess 220 formed within the elongate recess 180 in the sled-shaped finger holder 142. A locking ramp 222 across a front portion of the locking recess 220 guides the locking tip 214 into locking engagement with the sled shaped finger holder 142 when distally positioned (FIG. 5) to close and lock the upper jaw 114. The locking tip 214 is released when the thumb slide 208 is proximally positioned (FIG. 6).

It should be appreciated that the locking features may be reversed such that drawing the thumb slide aft effects closing and locking or that the upper jaw is locked open in addition to or as an alternative to locking closed. As another alternative, the jaws may be coupled to a lever to both pivot toward the other. As yet another alternative, the lever may translated one of the jaws toward the other with neither jaw being pivotally attached to the other, allowing for parallel orientation of the jaws.

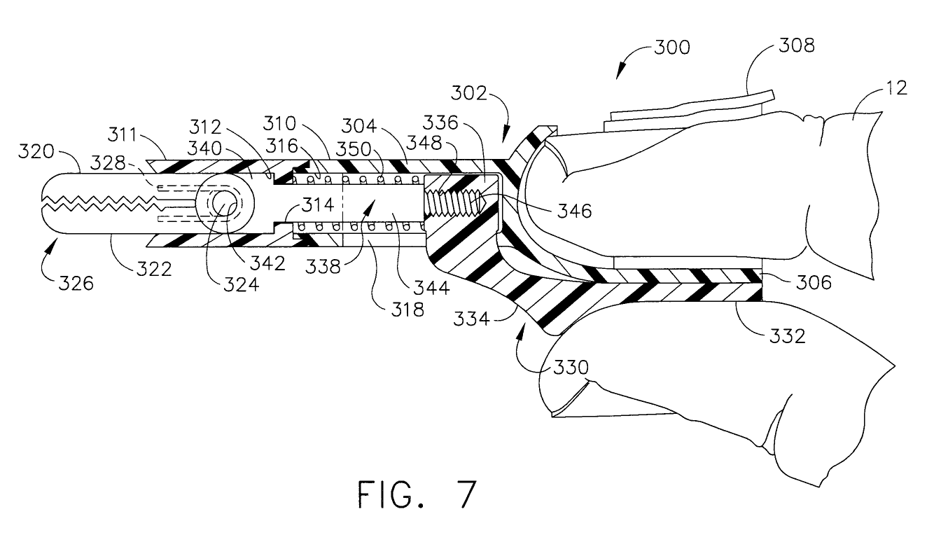

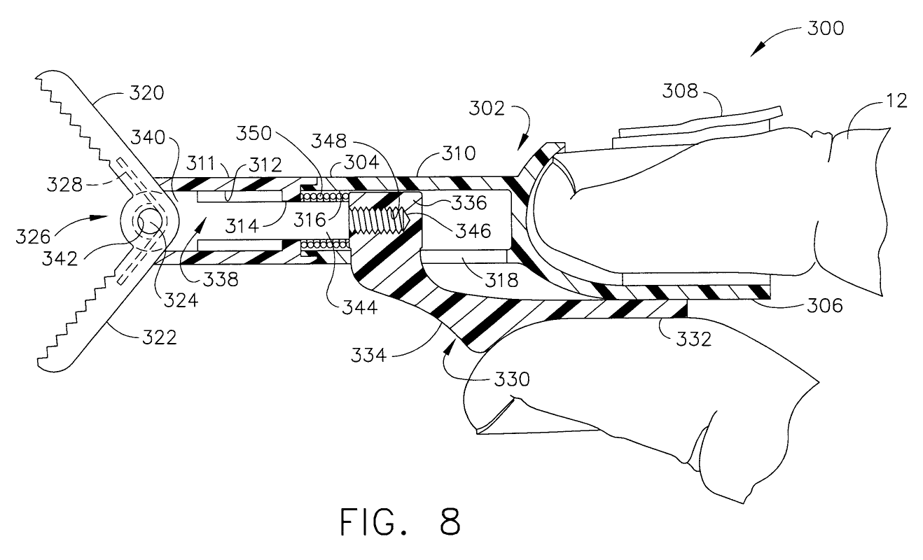

In FIGS. 7-8, an alternative fingertip surgical instrument (“extended end effector fingertip surgical instrument”) 300 is depicted as having a grasper or scissor end effector 302 that is actuated at a desired distance from the index finger 12 via an elongate neck 304. The index finger 12 is placed upon a finger holder 306 that is then held in place by a wing or band 308. It should be appreciated that the wing or band 308 may be formed from various types of adjustable attachment means that would be acceptable for surgical use and that securely grip the finger 12. A distally projecting neck cannula 310 extends from the finger holder 306. An outer sheath 311 with inwardly beveled outer edges is attached to a distal opening of the neck cannula 310 and defines a distal bore 312 that communicates and is longitudinally aligned through a narrow neck opening 314 at a proximal end of the outer sheath 311. The narrow neck opening 314 communicates and is longitudinally aligned with a proximal cylindrical spring cavity 316 defined by the neck cannula 310. A lower longitudinal actuator slot 318 passes through the neck cannula 310 to communicate with the cylindrical spring cavity 316.

An upper jaw 320 and lower jaw 322, which may have gripping, cutting, scissoring or other surfaces, rotate about a shared jaw axle 324 to form an end effector 326 sized to fit at least partially within the distal bore 312. A U-shaped clip spring 328 passes around the axle 324 and has an upper distal end attached to the upper jaw 320 and a lower distal end attached to the lower jaw 322 to bias the jaws 320, 322 to an open position (FIG. 8). A thumb slide actuator 330 has a thumb gripping portion 332 that underlies the finger holder 306 that is attached to a connecting portion 334 that moves within the lower longitudinal actuator slot 318 to a cylindrical portion 336 that is sized for movement within the proximal cylindrical spring cavity 316. A reciprocating shaft 338 is shaped with a bullet-shaped distal end 340 recessed on each side of an axle hole 342 to receive the jaws 320, 322 and to proximally ground against narrow neck opening 314 with a narrower rod 344 extending back through the cylindrical spring cavity 316 terminating in a screw portion 346 that is threaded into a threaded screw hole 348 in the cylindrical portion 336 of the thumb slide actuator 330. A compression spring 350 encompasses the narrower rod 344 and grounds against a proximal side of the narrow neck opening 314 and a distal side of the cylindrical portion 336 of the thumb slide actuator 330, creating a proximal bias on the end effector 326. When the bias withdraws the end effector 326 into the outer sheath, the opening bias on the end effector 326 is overcome and the jaws 320, 322 close (FIG. 7). The outer sheath 311 is assembled last to retain this spring 350. Distal movement of the thumb slide actuator 330 overcomes this bias to extend the end effector 326 out of the distal bore 312 to allow the end effector 326 to open (FIG. 8). Thus, coordinated movement of the entire fingertip surgical instrument 300 and the thumb slide actuator 330 allows manipulation or severing of internal tissue.

It should be appreciated that the configuration of the alternative fingertip surgical instrument 300 is illustrative and that applications consistent with the present invention may be biased open in the unactuated position. In addition, rather than both jaws actuating, one jaw may be fixed. Further, rather than relying upon a spring bias to pivot the jaws in one direction relative to each other, applications consistent with the present invention may include affirmative coupling with an actuator to transfer actuating motion to both open and to close the end effector. It should be appreciated that for clarity a straight and vertically aligned end effector is depicted, but the orientation of the end effector in applications consistent with the invention may include curved, longitudinal rotary and/or articulating structures for positioning prior to insertion or to remotely position the end effector after insertion. Further, for simplicity a fixed length elongated neck is depicted, but it should be appreciated that an adjustable (e.g., telescoping) portion of the elongate neck may be incorporated to adjust either prior to insertion or after insertion to a desired distance from the fingertip. It should be appreciated that the trigger may instead be a pivoting actuator whose motion is converted to a longitudinal reciprocating motion. As yet another alternative, the actuator may be slid an outer sheath over a fixed grasping portion rather than moving the grasping portion.

Internal Liquid Dispensers: Markers. Applications of an internal liquid dispensing instrument suitable for a HALS procedure are numerous. For instance, a fingertip ink marker may be used as a training tool to mark anatomic features. Another example is use as a planning tool to show where to make incisions, the path to follow, to establish orientation, and to cut profiles for feature alignment. As yet another example is use as a landmark identifier to avoid having to spend time relocating a structure. In addition to dispensing a marking liquid, other significant applications exist for the precise application of liquids as part of a HALS procedure, such fluid dispensers principally for but not limited to adhesives and sealants, with the dispensing of any fluid or gel chemistry for the additional purpose of drug delivery, barrier/scaffolding/buttress, or sclerosing/necrosing of tissue, with the application being of a permanent or temporary (time limited) nature. Dispersants may be self-contained or used with external activation sources such as moisture, oxygen (air) or lack of heat, light, etc. Applications may be surface, tissue to tissue or tissue to device in nature. Adhesives may be activated by moisture, peel-liner, or other delivery approaches. Absorbability of an ink, adhesive or physical marker conveyed as flowable material may be advantageous in certain applications. Mechanical forms may include a biocompatible collagen that has a tissue adhering adhesive.

Versions of a fingertip surgical instrument described hereafter thus include a fingertip mounting structure to which is attached a fluid containing structure that is selectively actuated to expose a fluid, liquid or gel (e.g., marking, adhesive, therapeutic compound) that is biocompatible and efficacious for application to internal tissue in a HALS procedure.

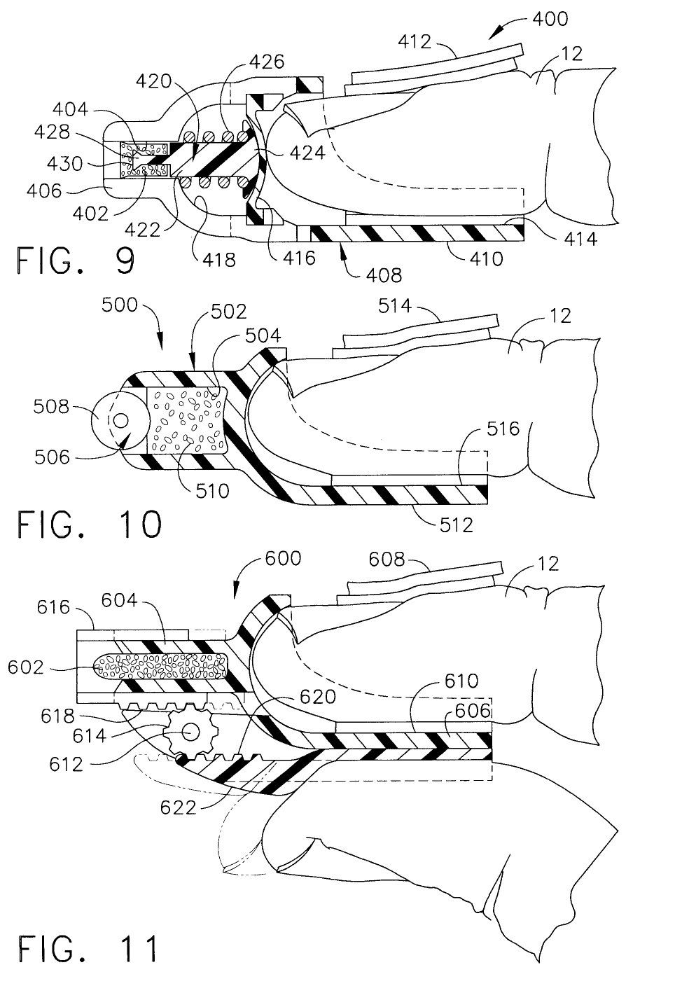

Returning to the drawings, in FIG. 9, yet an additional alternative fingertip surgical instrument (“extending inkpad fingertip instrument”) 400 includes a porous cylindrical ink dispensing component 402 held within a distal bore 404 of an end effector nozzle 406 formed in a finger holder 408. A proximal, finger portion 410 encompasses a lower and distal portion of the index finger 12, retained therein by a wing or band 412 attached to an upward, proximal surface 414 of the finger portion 410. It should be appreciated that the wing or band 412 may be formed from various types of adjustable attachment means that would be acceptable for surgical use and that securely grip the finger 12. A cylindrical diaphragm 416 is attached across a wider cavity 418 defined inside of the end effector nozzle 406 proximal to and communicating with the distal bore 404. A plunger 420 has a proximal shaft 422 sized to closely fit for translation within the distal bore 404 and has a rounded head 424 that contacts the diaphragm 416 from the distal side. A compression spring 426 larger than the diameter of the distal bore 404 encompasses the proximal shaft 422, urging the rounded head 424 proximally to position the diaphragm 416 into contact with the fingertip of the index finger 12. A small diameter plug attachment rod 428 extends distally from the proximal shaft 422 terminating in a flathead 430 embedded within the porous cylindrical ink dispensing component 402. Thus, when the end effector nozzle 406 is pressed against tissue, the fingertip of the index finger 12 depresses against the diaphragm 416, distally advancing the plunger 420 while compressing the compression spring 426, which in turn distally extends the porous cylindrical ink dispensing component 402 into contact with the internal tissue to impart a marking, adhesive and/or therapeutic effect, depending upon a flowable material absorbed by the ink dispensing component 402.

In FIG. 10, yet another alternative fingertip surgical instrument (“rollerball fingertip instrument”) 500 has a roller ball end effector 502 that includes a cylindrical reservoir 504 with a roller ball or disk 506 held for rotation and exposing a distal portion 508 to paint flowable material 510 drawn from the reservoir onto internal tissue (not shown). A finger mounting portion 512 is proximally attached to the cylindrical reservoir 504 to contact at least an undersurface and fingertip of the index finger 12, while a band or wing 514 attached to a proximal upward surface 516 of the finger mounting portion 512 retains the finger 12 in contact, accommodating a range of finger sizes. It should be appreciated that the wing or band 514 may be formed from various types of adjustable attachment means that would be acceptable for surgical use and that securely grip the finger 12. Thus, swiping contact with tissue causes the roller ball 506 to deposit flowable liquid 510 onto internal tissue. If a roller disk 506 is selected, a castor attachment may allow the roller disk 506 to readily align with the direction of swiping contact.

In FIG. 11, yet a further alternative fingertip surgical instrument (“sheathed marking fingertip instrument”) 600 has a marking element (e.g., ink soaked porous elongate rod) 602 partially embedded into a cylindrical end effector tip 604 attached to a finger mounting structure 606 that partially encompasses an index finger 12. It should be appreciated that the amount of flowable material may be augmented by a reservoir that communicates with the marking element 602 or may be limited to the quantity that the marking element 602 may absorb. A wing or band 608 attached to an upward, proximal surface 610 of the finger mounting structure 606 grips the finger 12. It should be appreciated that the wing or band 608 may be formed from various types of adjustable attachment means that would be acceptable for surgical use and that securely grip the finger 12. A horizontal gear axle 612 is attached to the finger mounting structure 606 below the cylindrical end effector tip 604 to support a vertically aligned spur gear 614. An outer cylindrical sleeve 616 encompasses the cylindrical end effector tip 604, longitudinally translating between a distal position encompassing the marking element 602 as shown and a proximal position exposing the marking element 602 depicted in phantom. A lower rack portion 618 formed on an exterior of the outer cylindrical sleeve 616 is in gear engagement to the spur gear 614, which in turn is in gear engagement to an upwardly presented gear rack portion 620 formed on a distal portion of a thumb slide 622, coupled for longitudinal translation to the finger mounting structure 606. Thus, distal movement of the thumb slide 622 causes the marking element 602 to be exposed for use. It should be appreciated that other mechanizations that tend to extend the marking element 602 or to withdraw a shielding component may be used consistent with aspects of the invention, to include but are not limited to a lever, flattening an arcuate member, etc.

In FIG. 12, an additional alternative fingertip surgical instrument (“direct coupled sheath marking fingertip instrument”) 700 has a marking element 702 partially embedded into a distally open cylindrical receptacle 704 of a thumb actuator 706. The marking element 702 may be manually drawn out of the receptacle 704 prior to use to expose a suitable portion, adjusting for the available longitudinal length of the marking element 702, especially for a marking element 702 that is mechanically rubbed off or dissolved by fluid. Alternatively, the marking element may comprise a porous structure that allows a flowable material retained therein to dispense upon contact. The cylindrical receptacle 704 translates within a distally open cylindrical end effector nozzle 708 that is attached to a finger mounting structure 710 that partially encompasses an index finger 12. A wing or band 712 attached to an upward, proximal surface 714 of the finger mounting structure 710 grips the finger 12. It should be appreciated that the wing or band 712 may be formed from various types of adjustable attachment means that would be acceptable for surgical use and that securely grip the finger 12. A lower longitudinal slot 716 along at least a proximal portion of the end effector nozzle 708 allows longitudinal movement of a connecting arm 718 that connects the receptacle 704 to a thumb contacting surface 720, forming the actuator 706. Distal movement of the actuator 706 exposes the marking element 702 for use distal to the end effector nozzle 708 (shown in phantom) and proximal movement of the actuator 706 hides the marking element 702 as depicted.

In FIG. 13, yet another alternative fingertip surgical instrument (“convex porous tip dispensing fingertip instrument”) 800 has a marking element 802 containing flowable material which is enclosed within a dispensing end effector 804. In particular, an outer cap 806 of the end effector 804 has a convex porous portion 808, which in the illustrative version is composed of a resilient material for deflection under a force exerted by a fingertip, and is presented centrally on an otherwise flat circular end 810, which in turn is attached to a distal cylindrical side wall 812 that transitions to a slightly smaller diameter proximal cylindrical side wall 814, presenting an internal locking lip 816 at the transition. A distally open cylindrical bore 818 of the end effector 804 has a diameter to closely fit within the proximal cylindrical side wall 814 and presents an outer locking lip 820 at its distal end that locks inside distal to the internal locking lip 816. A finger holder 822 is proximally attached to the distally open cylindrical bore 818 and partially encompasses an index finger 12. A wing or band 824, attached to an upward, proximal surface 826 of the finger holder 822, grips the finger 12. It should be appreciated that the wing or band 824 may be formed from various types of adjustable attachment means that would be acceptable for surgical use and that securely grip the finger 12. A spring post 828 extends distally from the finger holder 822 centered within the distally open cylindrical bore 818. A plunger 830 has a cylindrical side wall 832 sized to closely fit but translate within the distally open cylindrical bore 818 and spanned by a transverse seat disk 834 defining a shallow distal recess 836 that receives a smaller diameter base 838 of the marking element 802 that is otherwise sized to translate within the distally open cylindrical bore 818. The transverse seat disk 834 of the plunger 830 also defines a deeper proximal spring cavity 840 that receives a distal end of a compression spring 842 whose proximal end is received around the spring post 828. The spring biased plunger 830 maintains the marking element 802 in contact with an inner surface of the convex porous portion 808 of the outer cap 806 so that under cooperative deflection of the convex porous portion 808 that flowable material is forced out for application to internal tissue.

Internal Liquid Dispensing: Encapsulated Liquids. Some flowable materials to be dispensed may advantageously be encapsulated in ampoules or similar structures to preserve their properties until dispensing (e.g., a moisture or oxygen activated or two-part adhesive). An illustrative list of adhesives is contained in U.S. patent application Ser. No. 10/359,699 “Applicators, Dispensers And Methods For Dispensing And Applying Adhesive Material” to Goodman et al., filed 7 Feb. 2003, now published as U.S.2004/0190975A1 on 30 Sep. 2004, the disclosure of which is hereby incorporated by reference in its entirety.

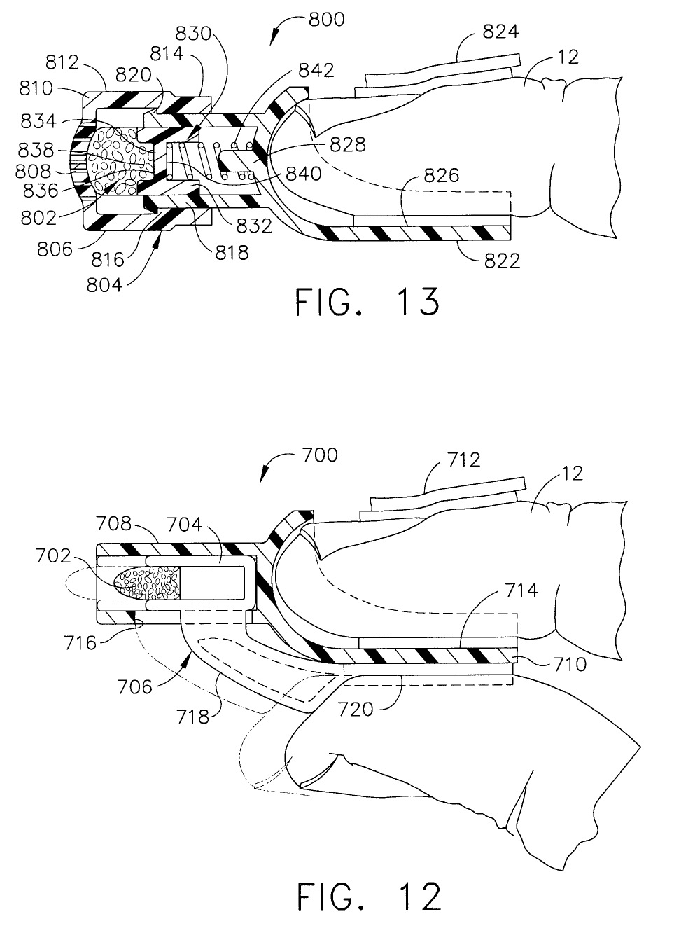

In FIG. 14, another alternative fingertip surgical instrument (“pushed ampoule dispensing fingertip instrument”) 900 has an ampoule 902 with distal scorings 904 whose proximal end is received within a distal recess 906 in a cylindrical pusher 908 of a thumb actuator 910. The cylindrical pusher 908 translates within a cylindrical end effector tube 912 that is attached to a finger holder 914 that partially encompasses an index finger 12. A wing or band 916 attached to an upward, proximal surface 918 of the finger holder 914 grips the finger 12. It should be appreciated that the wing or band 916 may be formed from various types of adjustable attachment means that would be acceptable for surgical use and that securely grip the finger 12. A lower longitudinal slot 920 along at least a proximal portion of the end effector tube 912 allows longitudinal movement of a connecting arm 922 that connects the pusher 908 to a thumb contacting surface 924, forming an actuator 926. A nozzle cap 928 has a wide diameter proximal ring 930 that fits over a distal portion of the end effector tube 912. The nozzle cap 928 includes a central bulbous portion 932 distally attached to the wide diameter proximal ring 930 and is sized to have an internal cavity 934 that continues the diameter of the end effector tube 912. A converging nozzle tip 936 is distally attached to the central bulbous portion 932. A spike member 938 internally received in the nozzle tip 936 extends an upper piercing arm 940 and a slightly shorter lower piercing arm 942 proximally toward the ampoule 902 in the internal cavity 934. It should be appreciated that the spike member 938 allows a longitudinal flow between the internal cavity 934 and an external orifice 944 of the nozzle tip 936. Distal movement of the actuator 926 impales the ampoule 902 upon the piercing arms 940, 942 of the spike member 938, filling the internal cavity 934 distal to the ampoule 902. Continued distal movement (shown in phantom) of the actuator 926 reduces the volume of the internal cavity 934, expelling the flowable material contents out of the external orifice 944 onto internal tissue.

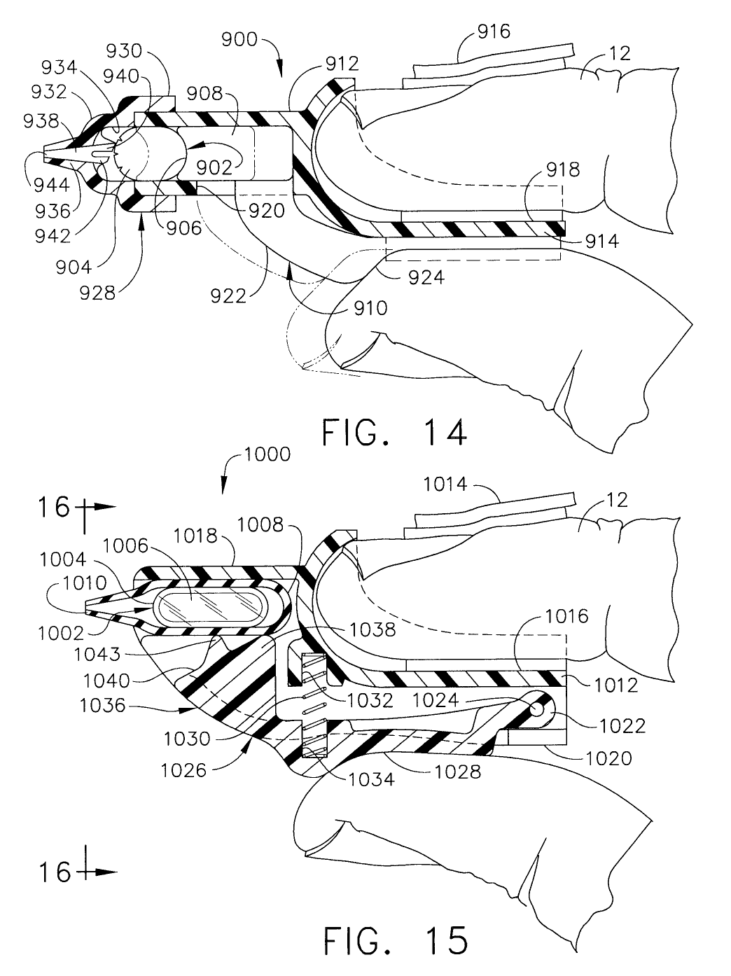

In FIGS. 15-16, yet a further alternative fingertip surgical instrument (“bottom ampoule crushing dispensing fingertip instrument”) 1000 has an ampoule 2 with an outer frangible elongate shell 1004 containing a flowable material 1006. The ampoule 1002 is contained within an elongate nozzle bulb 1008 have a converging nozzle orifice 1010 distally oriented. A finger holder 1012 receives and encompasses a distal and lower surface of an index finger 12, retained therein by a wing or band 1014 that is attached to a proximal upward surface 1016 of the finger holder 1012. It should be appreciated that the wing or band 1014 may be formed from various types of adjustable attachment means that would be acceptable for surgical use and that securely grip the finger 12. An end effector support portion 1018, having a uniform inverted U-shaped transverse cross section (FIG. 16) extends distally from the finger holder 1012 to encompass each side of the elongate nozzle bulb 1008 except distally and underneath. A U-shaped bracket 1020 extends below a proximal end of the finger holder 1012 to receive a proximal pivoting end 1022 of an actuator 1026 horizontally pinned therein by a pin 1024. A central portion 1028 of the actuator 1026 has a lower contour shaped for a thumb to rotate the actuator 1026 upwardly, resisted by a vertical compression spring 1030 whose top end is received in a downwardly open spring receptacle 1032 formed in the finger holder 1012 and an aligned upwardly open spring receptacle 1034 formed in the central portion 1028 of the actuator 1026. A distal portion 1036 of the actuator 1026 has a generally triangular vertical cross section (FIG. 15) and is laterally sized to closely fit for upward translation within the end effector support portion 1018. A top, proximal corner 1038 of a ramped surface 1040 of the distal portion 1036 initially makes nondeforming contact at an aft lower point on the elongate nozzle bulb 1008. Left, center and right upward bumps 1042, 1043, 1044 (FIG. 16) formed on the ramped surface 1040 initially make nondeforming contact to a longitudinal midpoint underneath the elongate nozzle bulb 8 respectively on a left, center and right side. It should be appreciated that upward actuation of the actuator 1026 causes the bumps 1042-1044 to fracture the elongate shell 1004 of the ampoule 1002 as the ramped surface 1040 progressively collapses a proximal portion of the elongate nozzle bulb 1008 to expel the flowable material 1006 out of the converging nozzle orifice 1010 until proximity with the end effector support portion 1018 and finger holder 1012 arrests further actuation.

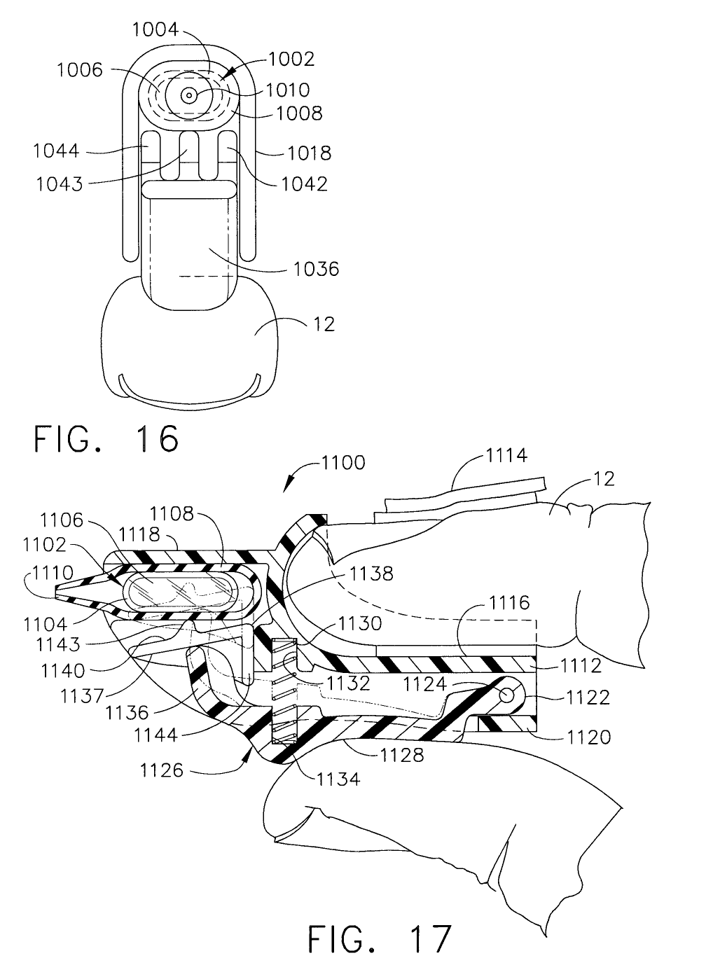

In FIG. 17, yet an additional alternative fingertip surgical instrument (“rocker bottom ampoule crushing dispensing fingertip instrument”) 1100 has an ampoule 1102 with a frangible elongate shell 1104 containing a flowable material 1106. The ampoule 1102 is contained within an elongate nozzle bulb 1108 have a converging nozzle orifice 1110 distally oriented. A finger holder 1112 receives and encompasses a distal and lower surface of an index finger 12, retained therein by a wing or band 1114 that is attached to a proximal upward surface 1116 of the finger holder 1112. It should be appreciated that the wing or band 1114 may be formed from various types of adjustable attachment means that would be acceptable for surgical use and that securely grip the finger 12. An end effector support portion 1118, having a uniform inverted U-shaped transverse cross section extends distally from the finger holder 1112 to encompass each side of the elongate nozzle bulb 1108 except distally and underneath. A U-shaped bracket 1120 extends below a proximal end of the finger holder 1112 to receive a proximal pivoting end 1122 of an actuator 1126 horizontally pinned therein by a pin 1124. A central portion 1128 of the actuator 1126 has a lower contour shaped for a thumb to rotate the actuator 1126 upwardly, resisted by a vertical compression spring 1130 whose top end is received in a downwardly open spring receptacle 1132 formed in the finger holder 1112 and an aligned upwardly open spring receptacle 1134 formed in the central portion 1128 of the actuator 1126. A distal portion of the actuator 1126 is a curved upward bar portion 1136 laterally sized to closely fit for upward translation within the end effector support portion 1118. A linearly-moved contact member 1137 has a rectangular plate surface 1140 whose midpoint rests upon the bar portion 1136 of the actuator 1126. A downturned flange 1144 at a top, proximal corner 1138 of the rectangular plate surface 1140 abuts a distal vertical, transverse surface of the downwardly open spring receptacle 1132 of the finger holder 1112. One or more upward bumps 1143 laterally arrayed across a longitudinal midpoint of the rectangular plate surface 1140 of the linearly-moved contact member 1137 are initially in nondeforming contact with a midpoint of an undersurface of the elongate nozzle bulb 1108. It should be appreciated that upward actuation of the actuator 1126 causes the linearly-moved contact member 1137 to move upwardly, maintaining a slight downward cant of its distal edge, causing bump(s) 1143 to fracture the elongate shell 1104 of the ampoule 1102 as the rectangular plate surface 1140 progressively collapses a proximal portion of the elongate nozzle bulb 1108 to expel the flowable material 1106 out of the converging nozzle orifice 1110 until proximity with the end effector support portion 1118 and finger holder 1112 arrests further actuation. Thus, the contact member 1137 linearly guided by the structure of the finger holder 1112 converts the rotation movement from the distal end 1136 of the actuator 1126 so that an optimized orientation of breaking and compressive contact may be imparted to the elongate nozzle bulb 1108 and ampoule 1102 throughout a desired distance of translation, even for a distal end 1136 that substantially changes its angular orientation.

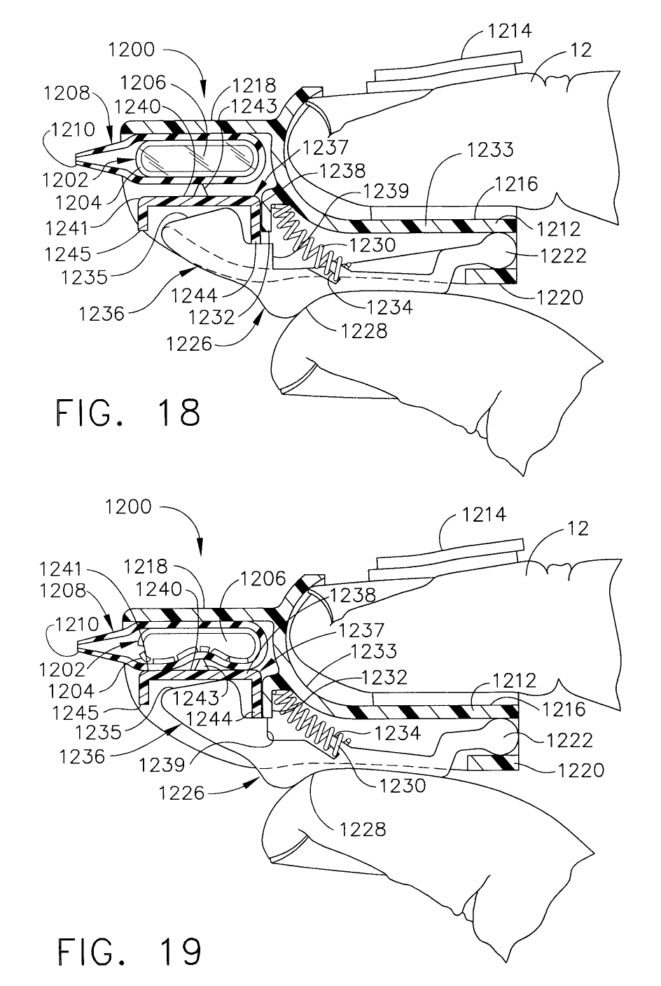

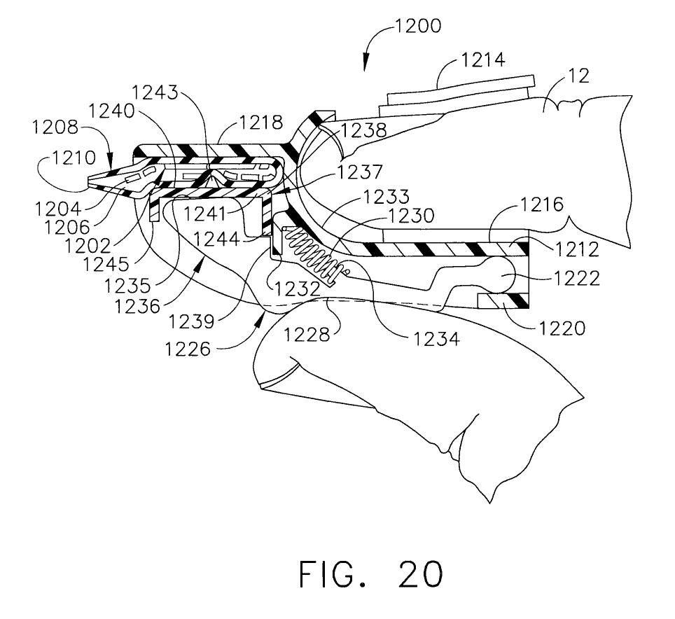

In FIGS. 18-20, another alternative fingertip surgical instrument (“two-step ampoule crushing dispensing fingertip instrument”) 1200 has an ampoule 1202 with a frangible elongate shell 1204 containing a flowable material 1206. The ampoule 1202 is contained within an elongate nozzle bulb 1208 having a converging nozzle orifice 1210 distally oriented. A finger holder 1212 receives and encompasses a distal and lower surface of an index finger 12, retained therein by a wing or band 1214 that is attached to a proximal upward surface 1216 of the finger holder 1212. It should be appreciated that the wing or band 1214 may be formed from various types of adjustable attachment means that would be acceptable for surgical use and that securely grip the finger 12. An end effector support portion 1218, having a uniform inverted U-shaped transverse cross section extends distally from the finger holder 1212 to encompass each side of the elongate nozzle bulb 1208 except distally and underneath. A U-shaped bracket 1220 extends below a proximal end of the finger holder 1212 to receive a proximal rod-shaped pivoting end 1222 of an actuator 1226. A central portion 1228 of the actuator 1226 has a lower contour shaped for a thumb to rotate the actuator 1226 upwardly (FIG. 18), then distally (FIG. 19), followed by more upward motion (FIG. 20) to sequentially rupture the ampoule and then to dispense the flowable material 1206 in a controlled rate. An angled compression spring 1230 has a top end attached to the finger holder 1212 proximal to a downward grounding flange 1232 extending from a finger shaped portion 1233 of the finger holder 1212. An upwardly and distally angled spring post 1234 formed in the central portion 1228 of the actuator 1226 receives a bottom end of the compression spring 1230 and aims toward the attachment of the top end of the compression spring 1230. A distal portion 1236 of the actuator 1226 has a vertical foot shape laterally sized to closely fit for upward translation within the end effector support portion 1218 and a slightly downwardly canted upper surface 1235. A crush detent step 1239 is formed on an upper transition between the distal portion 1236 and the central portion 1228 of the actuator 1226. A linearly-moved actuator member 1237 has a rectangular plate surface 1240 whose midpoint rests upon the bar portion 1236 of the actuator 1226. A downturned proximal flange 1244 at a top, proximal corner of the rectangular plate surface 1240 abuts a distal vertical, transverse surface presented by the downward grounding flange 1232 of the finger holder 1212. Thus, the structure of the finger holder 1212 guides the actuator member 1237 for linear movement to present an optimized breaking and contact surface to the nozzle bulb 1208 and ampoule 1202 through the rotational movement of the distal portion 1136 of the actuator 1126. A downturned distal flange 1245 from a top, distal corner of the rectangular plate surface 1240 resides in front of the distal portion 1236 of the actuator 1226. One or more upward bumps 1243 laterally arrayed across a longitudinal midpoint of the rectangular plate surface 1240 of the linearly-moved actuator member 1237 are initially in nondeforming contact with a midpoint of an undersurface of the elongate nozzle bulb 1208 (FIG. 18). Upward actuation of the actuator 1226 causes the linearly-moved actuator member 1237 to move upwardly, causing bump(s) 1243 to fracture the elongate shell 1204 of the ampoule 1202. The downturned grounding flange 1232 then contacts the crush detent step 1239, preventing any significant reduction in the volume of the elongate nozzle bulb 1208 (FIG. 19). When dispensing is then desired in the ensuing moments, the actuator 1226 may be distally moved slightly and then upwardly depressed to progressively collapse the elongate nozzle bulb 1208 to expel the flowable material 1206 out of the converging nozzle orifice 1210 (FIG. 20).

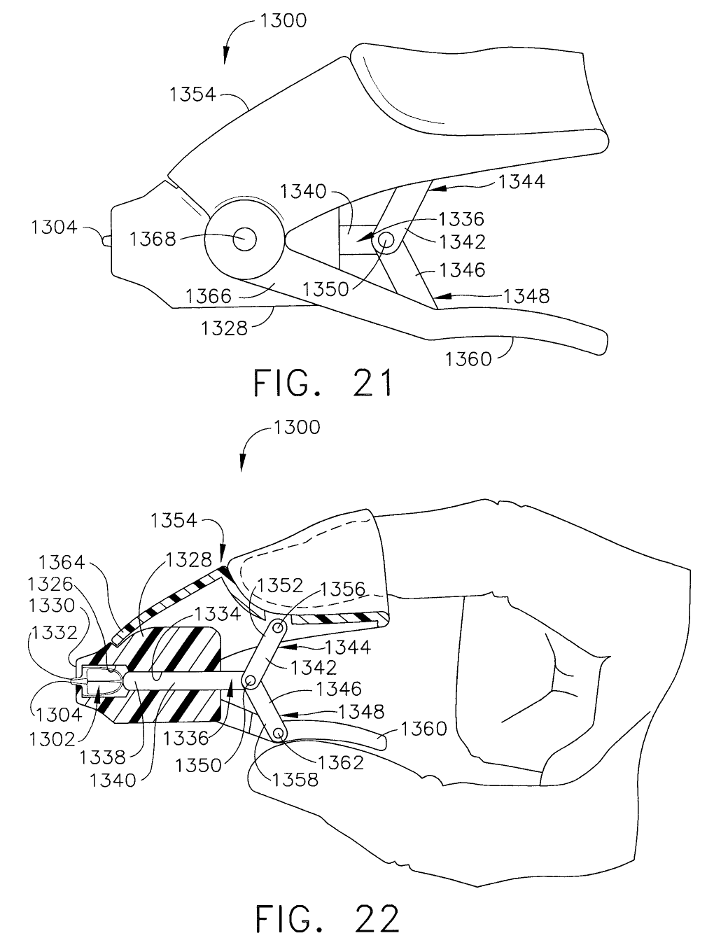

In FIGS. 21-24, an alternative fingertip surgical instrument (“necked ampoule applier”) 1300 receives a necked ampoule 1302 (FIG. 24) having a nozzle neck 1304 surrounded by a thick walled distal disk 1306 attached to an elongate capsule wall 1308 to form a reservoir 1310. For a dual chemical flowable material, in FIG. 24, the reservoir 1310 has a neck plug 1312 that seals off the nozzle neck 1304 and a fragile bifurcating barrier 1314 that separates a first flowable material 1316 from a second flowable material 1318. For a single chemical/mixture 1320, in FIG. 25, a necked ampoule 1302′ has a reservoir 1310′ filled with a single fragile neck barrier 1322′ plugging a nozzle neck 1304′, such as at a distal orifice end 1324′.

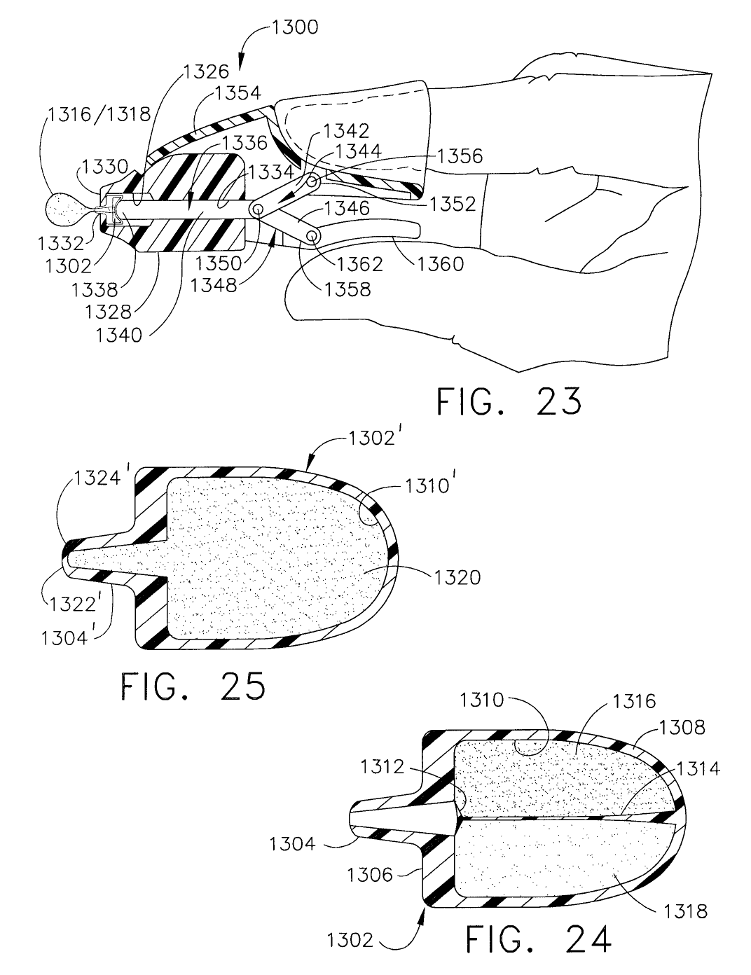

In FIGS. 21-23, an ampoule cavity 1326 sized for the necked ampoule 1302 is formed within an end effector block 1328. The ampoule cavity 1326 provides a distal circular nozzle surface 1330 against which the thick walled distal disk 1306 of the necked ampoule 1302 grounds and provides a centered nozzle hole 1332 in the nozzle surface 1330 through which the nozzle neck 1304 of the necked ampoule 1302 extends. A plunger passage 1334 communicates horizontally from a proximal direction through the end effector block 1328 to communicate with the ampoule cavity 1326. A rod plunger 1336 having a distal plunger end 1338 extends through the plunger passage 1334, initially in nondeforming contact with a proximal end of the necked ampoule 1302. A proximal plunger end 1340 of the rod plunger 1336 extends proximally out of the plunger passage 1334 of the end effector block 1328 a distance at least the longitudinal width of the necked ampoule 1302 (FIGS. 21-22). Both a lower end 1342 of an upper scissor link 1344 and an upper end 1346 of a lower scissor link 1348 are pivotally attached by a center rivet 1350 to the proximal plunger end 1340. An upper end 1352 of the upper scissor link 1344 is pivotally attached to a midpoint of an upper finger holder 1354 by an upper rivet 1356. A lower end 1358 of the lower scissor link 1348 is pivotally attached at a midpoint to a lower thumb actuator 1360 by a lower rivet 1362. Proximal ends 1364, 1366 of the upper finger holder 1354 and the lower thumb actuator 1360 are pivotally attached at about a longitudinal midpoint of the end effector block 1328 by a horizontal axle attachment 1368 (FIG. 21), forming an acute angle with one another bisected by the rod plunger 1336. Actuation of the upper finger holder 1354 and the lower thumb actuator 1360, as depicted in FIG. 23, causes the scissor links 1344, 1348 to rotate more toward the horizontal, translating the rod plunger 1336 distally, compressing the ampoule 1302 in a fashion to rupture the neck plug 1312 that seals off the nozzle neck 1304 and the fragile bifurcating barrier 1314 and to expel the flowable materials 1316, 1318 from the reservoir 1310 of FIG. 24 or to open the distal orifice end 1324′ of the necked ampoule 1302′ of FIG. 25.

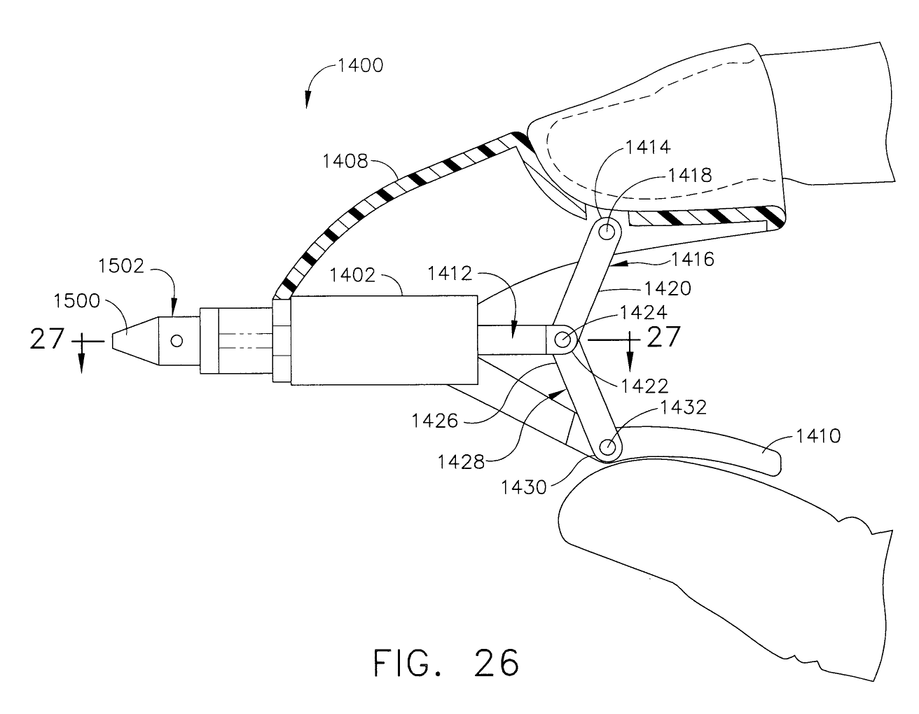

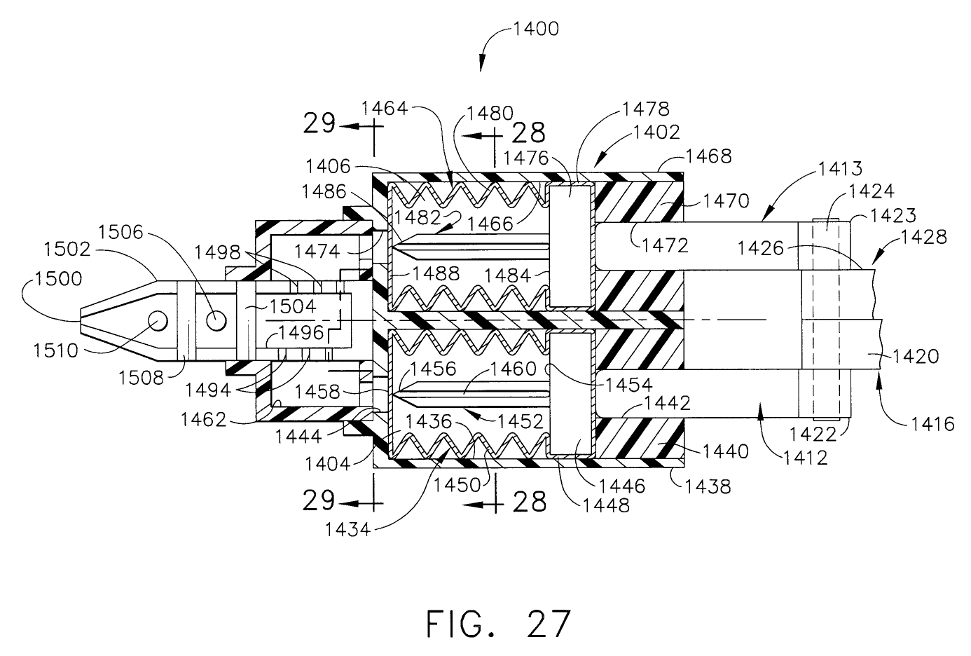

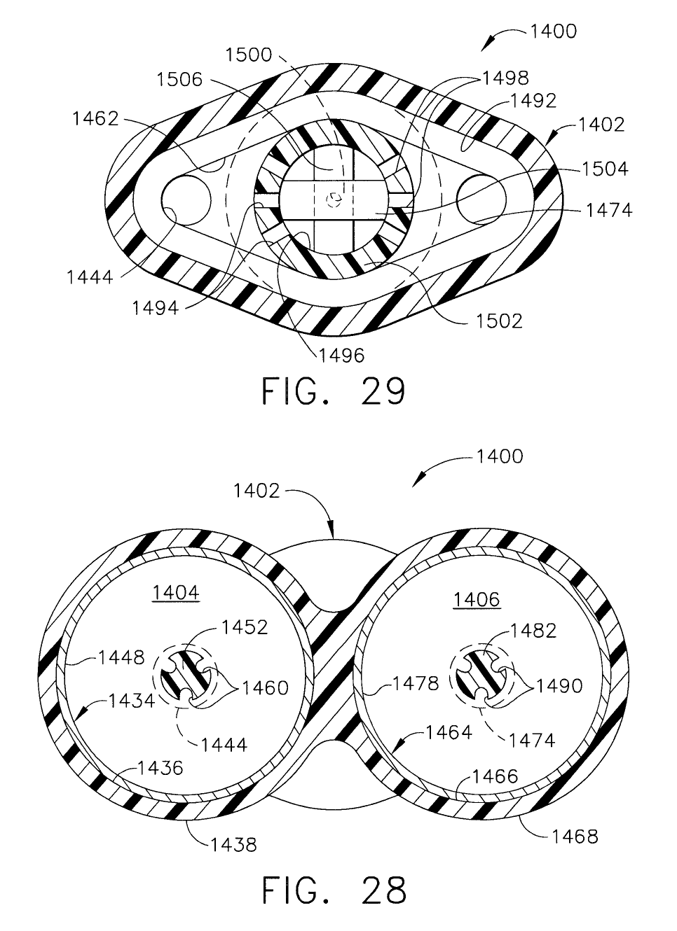

In FIGS. 26-31, yet another alternative fingertip surgical instrument (“two-component fingertip adhesive dispenser”) 1400 includes an end effector dispenser 1402 that internally mixes a portion of a first flowable material 1404 with a portion of a second flowable material 1406 for extended dispensing (FIG. 27). With particular reference to FIG. 26, dispensing is effected by squeezing an upper finger holder 1408 toward a lower thumb actuator 1410, both being distally, pivotally attached to the end effector dispenser 1402 (not shown). The motion is transferred to left and right rod plungers 1412, 1413 (FIG. 27) that extend proximally out of the end effector dispenser 1402. In particular, an upper end 1414 of an upper scissor link 1416 is pivotally attached to a midpoint of the upper finger holder 1408 by an upper rivet 1418. A lower end 1420 of the upper scissor link 1416 is pivotally attached to proximal ends 1422, 1423 of the left and right rod plungers 1412, 1413 by a center elongate rivet 1424. An upper end 1426 of a lower scissor link 1428 is also pivotally attached to the proximal ends 1422, 1423 of the rod plungers 1412, 1413 by the center elongate rivet 1424. A lower end 1430 of the lower scissor link 1428 is pivotally attached at a midpoint of the lower thumb actuator 1410 by a lower rivet 1432.

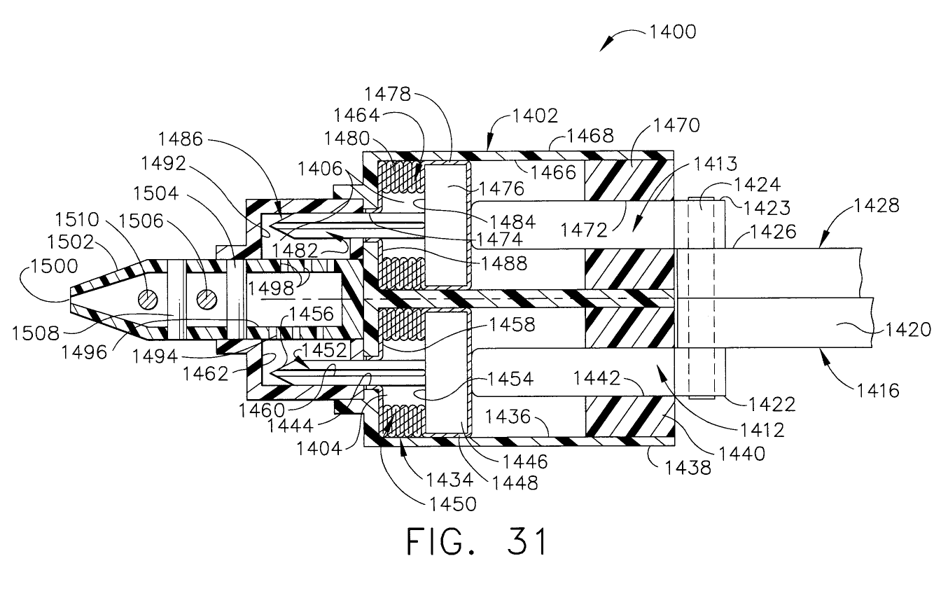

With particular reference to FIGS. 27-29, the first flowable material 1404 is contained within a left collapsible, generally cylindrical bladder 1434 sized to fit within a first cylindrical cavity 1436 formed within a left proximal cylindrical vessel 1438 of the end effector dispenser 1402. A left proximal solid portion 1440 of the left proximal cylindrical vessel 1438 includes a left rod passage 1442 which is longitudinally defined and receives the left rod plunger 1412 and has a left distal opening 1444 also longitudinally defined. A left cylindrical disk plunger 1446, laterally sized for the diameter of the first cylindrical cavity 1436, is retained within a proximal end 1448 aft of accordion sidewalls 1450 of the collapsible, generally cylindrical bladder 1434. A left generally cylindrical spike 1452 is attached to a longitudinal center of a distal surface 1454 of the left cylindrical disk plunger 1444 with a left sharp tip 1456 distally aimed longitudinally toward the left distal opening 1444 to pierce a distal wall 1458 of the bladder 1434. Three longitudinal aligned, radially spaced channel recesses 1460 (FIG. 29) formed along the length of the left generally cylindrical spike 1452 allow fluid material 1404 to flow out of the left cylindrical vessel 1438 into a left distal manifold chamber 1462 (FIG. 31).

The second flowable material 1406 is contained within a right collapsible, generally cylindrical bladder 1464 sized to fit within a second cylindrical cavity 1466 formed within a right proximal cylindrical vessel 1468 of the end effector dispenser 1402. A right proximal solid portion 1470 of the right proximal cylindrical vessel 1468 includes a right rod passage 1472 that is longitudinally defined and receives the right rod plunger 1413 and has a right distal opening 1474 also longitudinally defined. A right cylindrical disk plunger 1476, laterally sized for the diameter of the second cylindrical cavity 1466, is retained within a proximal end 1478 aft of accordion sidewalls 1480 of the collapsible, generally cylindrical bladder 1464. A right generally cylindrical spike 1482 is attached to a longitudinal center of a distal surface 1484 of the right cylindrical disk plunger 1476 with a right sharp tip 1486 distally aimed longitudinally toward the right distal opening 1474 to pierce a distal wall 1488 of the bladder 1464. Three longitudinal aligned, radially spaced channel recesses 1490 (FIG. 29) formed along the length of the right generally cylindrical spike 1482 allow fluid material 1406 to flow out of the right cylindrical vessel 1468 into a right distal manifold chamber 1492 (FIG. 31).

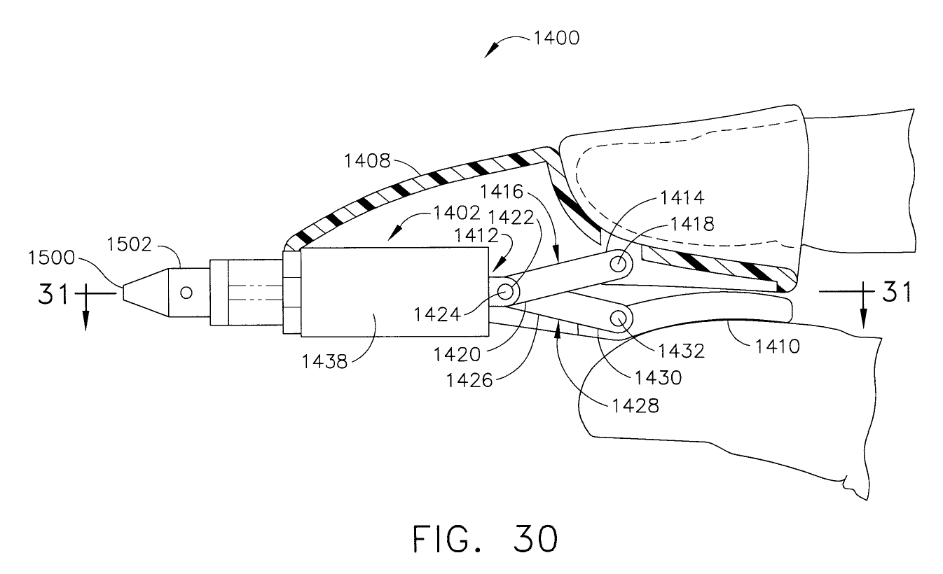

When the upper finger holder 1408 and lower thumb actuator 1410 are depressed toward each other as depicted in FIG. 30, the first fluid material 1404 is pushed out of the left distal manifold chamber 1462 through left inward holes 1494 into a central nozzle cavity 1496 and the second fluid material 1406 is pushed out of the left distal manifold chamber 1492 through right inward holes 1498 into the central nozzle cavity 1496. As the fluid materials 1404, 1406 are pushed toward a nozzle orifice 1500, a nozzle tube 1502 that defines the central nozzle cavity 1496 narrows. A proximal horizontal pin 1504, then a proximal vertical pin 1506, then a distal horizontal pin 1508, and finally a distal vertical pin 1510 pass through a longitudinal centerline of the nozzle tube 1502, longitudinally spaced from one another proximal to distal and alternatingly perpendicular to adjacent pins, to enhance turbulent, mixing interaction between the flowable materials 1404, 1406.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

While the present invention has been illustrated by description of several embodiments and while the illustrative embodiments have been described in considerable detail, it is not the intention of the applicants to restrict or in any way limit the scope of the appended claims to such detail. Additional advantages and modifications may readily appear to those skilled in the art.

For example, in addition to a roller ball or porous dispensing component or swab, liquid dispensing may be enhanced by adding other types of applicator tips (e.g., polymer loop, a spatula, a rolling ball, a grate, and a brush.

What is claimed is:

1. A fingertip-mounted minimally invasive surgical instrument comprising:
a first jaw comprising a concave contacting surface;
a second jaw comprising a convex contacting surface;
a finger mounting portion supporting the first and second jaws; and
a finger actuated member supported for movement by the finger mounting portion and connected to a selected one of a group consisting of the first and second jaw to effect closure to a spacing sufficient to orient and grip a suture needle.

2. The fingertip-mounted minimally invasive surgical instrument of claim 1, wherein the finger actuated member comprises a pivotally attached lever connected via a link to a proximal pivot portion of the selected one of the first and second jaw, the other one of the first and second jaw attached to the finger mounting portion.

3. The fingertip-mounted minimally invasive surgical instrument of claim 2, further comprising a locking member attached for movement to a selected one of a group consisting of the finger mounting portion and the lever to selectively engage the other one to maintain the jaws in a closed state.

4. The fingertip-mounted minimally invasive surgical instrument of claim 3, wherein the locking member comprises a thumb slide having a slide portion positioned to slide along a selected surface of the lever and having a locking tip connected to the slide portion positioned to reciprocate within a recess in the finger mounting portion that terminates in a locking recess.

5. A fingertip-mounted minimally invasive surgical instrument comprising:
a finger mounting portion;
pair of pivotally attached jaws;
an elongate neck spacing the pair of pivotally attached jaws from the finger mounting portion; and
a finger actuated member coupled for movement to the finger mounting portion and to the pair of pivotally attached jaws to effect opening and closing of the pair.

6. The fingertip-mounted minimally invasive surgical instrument of claim 5, further comprising:
a clip spring attached to the pair of pivotally attachment jaws imparting an opening bias; and
an elongate outer sheath attached to the finger mounting portion and encompassing the elongate neck that is connected between the pivotal attachment between the jaws and the finger actuated member;
wherein the finger actuated member is positionable distally to extend the pair of jaws out of the sheath for opening and proximally to draw a portion of the jaws into the sheath for closing.

7. A fingertip-mounted minimally invasive surgical instrument comprising:
a pair of grasping jaws;
a finger mounting portion supporting the first and second jaws;
a finger actuated member supported for movement by the finger mounting portion operable to open and to close the jaws; and
a locking member positionable to maintain the finger actuated member in a selected one of a group consisting of a jaws open state and a jaws closed state.

8. The fingertip-mounted minimally invasive surgical instrument of claim 7, wherein the locking member comprises a thumb slide having a slide portion positioned to slide along a selected surface of the lever and having a locking tip connected to the slide portion positioned to reciprocate within a recess in the finger mounting portion that terminates in a locking recess.

9. The fingertip-mounted minimally invasive surgical instrument of claim 8, wherein the finger actuated member is attached for pivotal movement toward the finger actuated member to effect the selected state, the locking recess holding the lever proximate to the finger actuated member when engaged.
